(19) United States
(12) Patent Application Publication
(10) Pub. No.: US 2015/0265228 A1
(43) Pub. Date: Sep. 24, 2015

(54) Method for Registering a Near-Infared Spectroscopy Map and an Anatomical Image Data Record and X-Ray Device

(71) Applicant: Yiannis Kyriakou, Spardorf (DE)

(72) Inventor: Yiannis Kyriakou, Spardorf (DE)

(21) Appl. No.: 14/660,213

(22) Filed: Mar. 17, 2015

(30) Foreign Application Priority Data
Mar. 21, 2014 (DE) 102014205313.7

(51) Int. Cl. A61B 6/00; A61B 5/00; A61B 5/1455

(52) U.S. Cl. CPC A61B 6/4417; A61B 5/14553; A61B 6/481; A61B 5/0075; A61B 6/487; A61B 6/507; A61B 6/5247; A61B 6/463

(57) ABSTRACT

The embodiments relate to registering a near-infrared spectroscopy map and an anatomical image data record of a target region of the human body. A near-infrared spectroscopy data record of the target region is recorded using a multichannel near-infrared spectroscopy device including a plurality of sensor elements in a sensor arrangement. The near-infrared spectroscopy data record is analyzed to produce a near-infrared spectroscopy map. A three-dimensional anatomical image data record is recorded using an X-ray device without either sensor arrangement or target region being moved in comparison with the recording of the near-infrared spectroscopy data record. The sensor elements are segmented and localized in the anatomical image data record. The near-infrared spectroscopy map and the anatomical image data record are registered on the basis of the known positions of the sensor elements relative to the near-infrared spectroscopy map and in the anatomical image data record.

### CROSS-REFERENCE TO RELATED APPLICATIONS

This application claims the benefit of DE 10 2014 205 313.7, filed on Mar. 21, 2014, which is hereby incorporated by reference in its entirety.

### TECHNICAL FIELD

The embodiments relate to a method for registering a near-infrared spectroscopy map and an anatomical image data record of a target region of the human body, (e.g., a head), and to an X-ray device.

### BACKGROUND

Near-infrared spectroscopy is a spectroscopic method that works in the near-infrared range of the electromagnetic spectrum, e.g., at wavelengths of approximately 800 nm to 2,500 nm. One of the preconceptions of the near-infrared radiation used is that it penetrates far into an object to be measured. When originally used, near-infrared spectroscopy was employed primarily for the testing of materials. However, it was then proposed that near-infrared spectroscopy (NIRS) may also be employed for the purpose of measuring the human body. In this case, advantage was taken of the fact that the transmission and absorption of the near-infrared light in tissues of the human body contains information about the hemoglobin concentration and/or a change thereof. In particular, one use of near-infrared spectroscopy in this case is to produce oxygenation maps of the human brain, which maps may therefore describe the flow of blood, or to check the brain function in other non-invasive ways, since the near-infrared light may penetrate the human skull without problem and may therefore measure the flow of blood in the brain. In order to obtain positional resolution, multichannel near-infrared spectroscopy (MNIRS) has also been proposed, including a plurality of sensor elements with a transmitter and a receiver in a sensor arrangement. Each sensor element may be assigned to a specific region of the human brain (or other target region in the human body) in this case, such that, e.g., the local oxygenation of superficially situated regions of the brain may be detected.

This non-invasive technology extends the range of functional diagnostics in the field of neurology, and is also suitable for characterizing disruptions in the cerebral flow of blood under clinical conditions and in combination with other, already established examination methods such as ultrasound, for example. Known research projects are therefore already using multichannel near-infrared spectroscopy to produce maps of the cortical blood flow in the brain for typical and frequently occurring vascular pathologies, in order to detect impaired blood flow where therapy may be relevant in patients, and possibly to apply an appropriate therapy.

The processing of near-infrared spectroscopy data to produce a near-infrared spectroscopy map, which may be two-dimensional or three-dimensional, is possible if the relative positions of the sensor elements that receive data are known relative to each other. The individual sensor element may then be assigned a region to which its data applies, wherein methods that allow interpolation between sensor elements are also readily conceivable. A two-dimensional near-infrared spectroscopy map is produced because the sensor arrangements, such as in the form of a hood in the case of brain examinations, are viewed with reference to the measuring surface. Three-dimensional near-infrared spectroscopy maps, which take into account the complete three-dimensional arrangement of the sensor elements relative to each other, are naturally also conceivable.

Near-infrared spectroscopy is problematic in that it does not depict any anatomical structures. This provides that the assignment to anatomical structures may at best be roughly assessed with reference to the known position of the sensor element on the patient. However, more precise anatomical assignments are not possible. In particular, this is relevant if the near-infrared spectroscopy is to be employed in a functional manner for the purpose of monitoring surgical interventions, (e.g., minimally invasive interventions in the brain). It is then extremely difficult to assign the measured near-infrared spectroscopy data to specific anatomical regions or structures. Therefore, X-ray perfusion measurements may be employed today for the purpose of monitoring and checking the success of therapy, particularly in the case of minimally invasive interventions in the human brain. These measurements expose the patient to high levels of radiation, however, and require an interruption of the intervention. Although near-infrared spectroscopy may be a substitute for the perfusion measurement in functional terms, the lack of anatomical reference nonetheless presents a problem.

### SUMMARY AND DESCRIPTION

The scope of the present invention is defined solely by the appended claims and is not affected to any degree by the statements within this summary. The present embodiments may obviate one or more of the drawbacks or limitations in the related art.

The object of the embodiments is to specify a possibility that is easy to realize, particularly in the context of minimally invasive interventions in the brain, by which anatomical information and near-infrared spectroscopy maps may be related to each other.

In order to achieve the object, provision is made for a near-infrared spectroscopy data record of the target region to be recorded using a multichannel near-infrared spectroscopy device including a plurality of sensor elements in a sensor arrangement and, using spatial information relating to the sensor elements and an assignment of near-infrared spectroscopy data to the sensor element that recorded the near-infrared spectroscopy data. Provision is also made to analyze the near-infrared spectroscopy data to produce a near-infrared spectroscopy map. Provision is further made for a three-dimensional anatomical image data record to be recorded using an X-ray device without moving either sensor arrangement or target region in comparison with the recording of the near-infrared spectroscopy data record, at least some of the sensor elements being visible in the three-dimensional anatomical image data record. Provision is further made for the sensor elements are segmented and localized in the anatomical image data record. Finally, provision is further made to register the near-infrared spectroscopy map and the anatomical image data record on the basis of the known positions of the sensor elements relative to the near-infrared spectroscopy map and in the anatomical image data record.

The high-resolution X-ray imaging and the functional multichannel near-infrared spectroscopy are therefore combined in order that the functional near-infrared spectroscopy data may be associated with anatomical features. While the near-infrared spectroscopy is not able to support a high-resolution representation of anatomical structures, the two-dimensional and three-dimensional X-ray imaging offers precisely this possibility, with the advantageous finding that the sensor elements still arranged in the target region may be depicted extremely well and therefore segmented in the X-ray imaging. Since the positions of the sensor elements determine the structure of the near-infrared spectroscopy map, but may also be detected in the anatomical image data record, it is therefore possible to associate these positions and obtain a registration between the system of coordinates of the near-infrared spectroscopy map and that of the X-ray device. As a result of the different geometric representation that is often used for multichannel infrared spectroscopy data, a flexible registration applies in most cases, providing that a non-rigid transformation is provided between the system of coordinates of the near-infrared spectroscopy maps and that of the X-ray device. In the context of the registration, it is also advantageous that a large number of sensor elements is used for the multichannel near-infrared spectroscopy, most of which are situated in a specific arrangement relative to each other, (e.g. when using a hood or similar), thereby further simplifying the registration by virtue of the many positions that are known to both systems of coordinates.

The method therefore makes it possible to establish and represent a combined data record, for example, by at least partially amalgamating the near-infrared spectroscopy map with the anatomical image data record. In this way, it is possible to generate representations that depict the near-infrared spectroscopy data in the anatomical context.

The method may be employed particularly appropriately in the context of a surgical intervention, and therefore the monitoring of a surgical intervention in particular, and this is discussed in greater detail below with reference to fluoroscopic monitoring. In fact, if multichannel near-infrared spectroscopy recordings are performed repeatedly in particular, a method is produced that allows the influence of the intervention on the target region to be understood in real time. This provides that current functional data may be obtained readily and repeatedly from the target region during the intervention itself, particularly if the sensor arrangement does not move relative to the target region. The data may be displayed relative to the anatomy, (e.g., in the combined representation), by virtue of the registration with the anatomical image data record. It is therefore beneficial to establish at least one further current near-infrared spectroscopy map during and/or after the surgical intervention, and to update the combined data record on the basis of the current near-infrared spectroscopy map. In this way, the near-infrared spectroscopy provides a true substitute for an X-ray perfusion measurement that may not reasonably be integrated into an ongoing intervention. The relevant person is therefore given real-time control over the surgical intervention by feedback relating to the function (near-infrared spectroscopy map).

In terms of functionality, the application of such combined data records or the real-time monitoring of an intervention is particularly advantageous if the target region includes the brain of a patient and an oxygenation map is determined as a near-infrared spectroscopy map. As mentioned above, multichannel near-infrared spectroscopy provides a method of determining the brain oxygenation in real time, and consequently also determining the influence of an intervention on the brain oxygenation and hence blood flow. In this case, the near-infrared spectroscopy map depicts the cortical oxygenation as a substitute for a perfusion measurement.

According to an advantageous embodiment, the segmentation of the sensor elements is based on threshold values and/or makes use of prior knowledge, in particular, relating to the geometry and/or attenuation characteristics and/or the relative arrangement of the sensor elements. Since sensor elements may contain high-attenuation materials, (e.g., metallic components), the sensor elements stand out in the anatomical image data record. Therefore, it is advantageous to employ a segmentation based on threshold values, this being easy to realize. In this case, it is also possible to take prior knowledge into consideration that relates not only to the aforementioned attenuation characteristics, but also to the geometry and/or the relative arrangement of the sensor elements, as this may also be applicable in the context of a validation check within the segmentation. Prior knowledge may be used to configure the threshold value, restrict the search region, etc. Specific segmentation algorithms that may be used are also known and do not require further explanation here.

In a particularly advantageous embodiment, image monitoring is performed for an intervention in a patient. The intervention is minimally invasive, in particular, wherein two-dimensional fluoroscopic images of the target region are recorded by the X-ray device. With reference to the registration between the anatomical image data record and the near-infrared spectroscopy map, a registration between the near-infrared spectroscopy map and the fluoroscopic images is established for the purpose of a combined information representation. Additionally, in particular, at least some of the near-infrared spectroscopy data is superimposed on the fluoroscopic image in the representation. Therefore, if the same spatial arrangement and the same X-ray device are used to record fluoroscopic images for monitoring the intervention, the registration may be readily transferred to the fluoroscopic images of the X-ray device, wherein it is also conceivable first to compute a registration between the anatomical image data record and the fluoroscopic images, which still makes it possible then to infer a transformation from the system of coordinates of the near-infrared spectroscopy map into the system of coordinates of the fluoroscopic images, and hence into the current system of coordinates of the X-ray device. It is therefore possible to superimpose near-infrared spectroscopy data onto the fluoroscopic images, which depict, e.g., an instrument used during the intervention, in order to set this information into actual context for the intervention.

In this connection, a particularly useful development provides for further recordings of further current near-infrared spectroscopy data to be made repeatedly using the sensor arrangement, and for the current near-infrared spectroscopy data to be used in the information representation in each case. If the live fluoroscopic images are represented with live near-infrared spectroscopy maps or parts thereof, the person performing the intervention may observe the influence of their measures, (e.g., vascular recanalization), on the local blood flow in real time. For example, the current cortical oxygenation may be measured during an intervention in the brain and represented in a manner that is superimposed on the current fluoroscopic images.

At least some of the anatomical data may also be used in the information representation. This provides that the procedure that may be known from the art may also be employed here to integrate anatomical structures, (which are possibly not clearly visible enough in the fluoroscopic images), into the information representation, in particular, by representing at least some of the anatomical image data or data derived therefrom in a manner that is superimposed on the fluoroscopic image.

In certain embodiments, the fluoroscopic images are recorded without any movement of the sensor arrangement relative to the patient and without any movement of the patient relative to the X-ray device, such that the validity of the original registration between the anatomical image data record and the system of coordinates that is defined by the sensor arrangement is maintained. In reality, it is however possible that (e.g., small) relative movements will occur.

In this connection, a particularly advantageous embodiment provides for the sensor elements to be segmented in the fluoroscopic images also, and analyzed with regard to registration errors, in particular, caused by movement. Since the sensor elements will already be identifiable in the X-ray imaging of the anatomical image data record, it is assumed that they will also be easy to identify in the fluoroscopic images and may therefore be segmented there likewise. By virtue of the registration between the anatomical image data record and the near-infrared spectroscopy map, e.g., the system of coordinates of the X-ray device for recording the anatomical image data record and the system of coordinates that is defined by the sensor arrangement and forms the basis of the near-infrared spectroscopy map, there exist expected values, where the sensor elements may be visible in the fluoroscopic images. Deviations from these reference positions therefore represent an indication of a registration error (which may be due to a movement) and may be analyzed accordingly. In this sense, the position of the sensor elements ultimately serves as a “movement tracker”, and therefore registration errors serve as indicators of a movement, of the patient in particular.

In this connection, it is particularly useful for an automatic correction of the registrations to be performed on the basis of the segmented sensor elements in the fluoroscopic image, and/or for a warning notification to be output to a user if a deviation from the registration exceeds a threshold value. The positions of the sensor elements in the fluoroscopic image, specifically their deviation from the expected position based on the registration, may therefore be used to update and therefore correct the registration. In this way, movement compensation may be achieved by tracking the sensor elements in the fluoroscopic images. If the deviations are so great that reliable correction of the registration is no longer possible, this may be communicated via a warning notification to a user, who may then record a new three-dimensional anatomical image data record if necessary, in order to update the registration, for example.

The anatomical image data record may be registered with at least one further image data record, in particular, a previously recorded image data record or an image data record that has been recorded (e.g., by another device), wherein a registration between the further image data record and the near-infrared spectroscopy map is established using the registration between the anatomical image data record and the near-infrared spectroscopy map. In addition to fluoroscopic images, the anatomical image data record may therefore be used to create a registration with a multiplicity of further image data records, in order to associate the information contained therein with the information of the near-infrared spectroscopy map, in order therefore, e.g., to generate and/or supplement superimposed representations in particular. The further image data records may be, e.g., preoperative planning and/or diagnostic image data records in the case of an intervention, though other data sources used during an intervention may also be included.

Specifically, a further image data record may be an image data record of the digital subtraction angiography and/or a perfusion image data record, in particular, as recorded using the X-ray device. Regarding interventions in the blood vascular system of a patient in particular, it may be appropriate also to make use of digital subtraction angiography, thereby obtaining an image of the blood vascular system that is as anatomically accurate as possible, and which may then be combined with the flow of blood if a registration with recorded near-infrared spectroscopy maps is available. Perfusion measurements, particularly from the same X-ray device that recorded the anatomical image data record, may advantageously supplement the functional data of the near-infrared spectroscopy map if the near-infrared spectroscopy is not already being used as a sole substitute for the perfusion measurement.

The further image data records need not necessarily be recorded using the X-ray device, and is it possible to conceive of a multiplicity of further image data records that may be registered, e.g., by anatomical features with the anatomical image data record, and therefore with the near-infrared spectroscopy map. Examples include magnetic resonance image data records, ultrasound image data records, computer tomography image data records, etc.

Provision may also be made for establishing a four-dimensional near-infrared spectroscopy map as a result of recording near-infrared spectroscopy data at various time points. It is then possible, for example, to supplement static X-ray recordings, in particular, the anatomical image data record, with four-dimensional functional information, in particular, relating to perfusion. In this way, temporal sequences are also illustrated and the information basis is therefore improved.

Use may be made of an X-ray device including an integrated near-infrared spectroscopy sensor arrangement. In this way, the complete multichannel near-infrared spectroscopy device may also be integrated advantageously into the X-ray device. This produces a combined device that allows both X-ray imaging and near-infrared spectroscopy and, by virtue of the method, allows an information amalgamation of the data that is recorded in each case, since this may be viewed in the same system of coordinates by virtue of the registration.

In this connection, it is also conceivable for the sensor arrangement to be arranged in a known spatial relationship to an X-ray source and an X-ray detector of the X-ray device, in particular, by a designated fixing the X-ray device. The registration of the systems of coordinates of the X-ray device and the near-infrared spectroscopy maps that are determined by the sensor arrangement may then be established in the context of a calibration measurement and used for subsequent diagnostic measurements. The anatomical image data record that is used to create the registration need not therefore necessarily contain anatomical features of a patient, but merely needs to depict the sensor arrangement. Since an anatomical image data record may be recorded in any case, in particular, by rotating a C-arm about the patient, in order to provide the anatomical information that is also used for superimposition in the case of fluoroscopic imaging or the like, the aforementioned calibration may obviously be performed as part of preparations for each measurement. The known spatial relationship of the sensor arrangement to the X-ray source and the X-ray detector may also be used to increase the reliability of the registration process by including this knowledge.

In this case, the sensor arrangement of the multichannel near-infrared spectroscopy device may be integrated with the X-ray device in a head shell of a patient couch, for example. In this way, a patient is so held in place as to have a constant contact with the head shell and the signal transfer is not disrupted. In cases where the sensor elements of the sensor arrangement are not visible in the X-ray image, (e.g., in fluoroscopic images), provision may be made for the head shell itself and/or markers on the head shell to be so positioned as to be continuously visible in the X-ray recordings, of the brain in this case, such that the system of coordinates of the head shell and hence the sensor arrangement may be monitored at all times, which may be relevant for tracking the patient movement as explained above. This provides that there is a fixed connection between the head shell, the sensor elements, and the markers.

Provision may therefore be made for arranging at least one marker, which is visible in the anatomical image data record and/or the fluoroscopic images, in a fixed geometrical relationship to the sensor elements, in particular, on a mounting for the sensor arrangement. The marker is taken into consideration when establishing the registration between the anatomical image data record and the near-infrared spectroscopy map and/or when checking for registration errors. In this case, such a mounting for the sensor arrangement, (e.g., the head shell), also allows the sensor arrangement, possibly with the mounting, to be removed for examinations in which the near-infrared spectroscopy is not required.

In addition to the method, the present embodiments also relate to an X-ray device including a control device for performing the method. All of the explanations relating to the method apply analogously to the X-ray device, and this therefore has the advantages cited above. It is therefore particularly appropriate for the multichannel near-infrared spectroscopy device with the sensor arrangement to be integrated into the X-ray device, wherein the control device may be so designed as to control the recording operation for both the X-ray data and the near-infrared spectroscopy data.

The X-ray device may moreover be designed as a C-arm X-ray device, which therefore has a C-arm on which an X-ray source and an X-ray detector are arranged facing each other. Such C-arm X-ray devices are outstandingly effective when employed in the context of a minimally invasive intervention, in particular, since the recording arrangement including the X-ray detector and the X-ray source may be removed from the intervention region by virtue of the movement possibilities. If the near-infrared spectroscopy device is integrated into the X-ray device, such an X-ray device offers an outstanding assistance when performing minimally invasive interventions in the blood vascular system of the brain. Three-dimensional recordings may be produced using such an X-ray device by rotating the C-arm about the target region, for example, wherein two-dimensional projection images are recorded from different projection directions and may then be used to reconstruct a three-dimensional image data record in a manner that is known. This method is also referred to by the term “DynaCT” in the prior art.

### BRIEF DESCRIPTION OF THE DRAWINGS

FIG. 1 depicts a flowchart of an exemplary embodiment of the method.

FIG. 2 depicts a schematic representation of an embodiment of an anatomical image data record.

FIG. 3 depicts a schematic representation of an embodiment of a near-infrared spectroscopy map.

FIG. 4 depicts a schematic representation of an embodiment of a combined data record.

FIG. 5 depicts an embodiment of an information representation during a minimally invasive intervention.

FIG. 6 depicts a schematic diagram of an embodiment of an X-ray device.

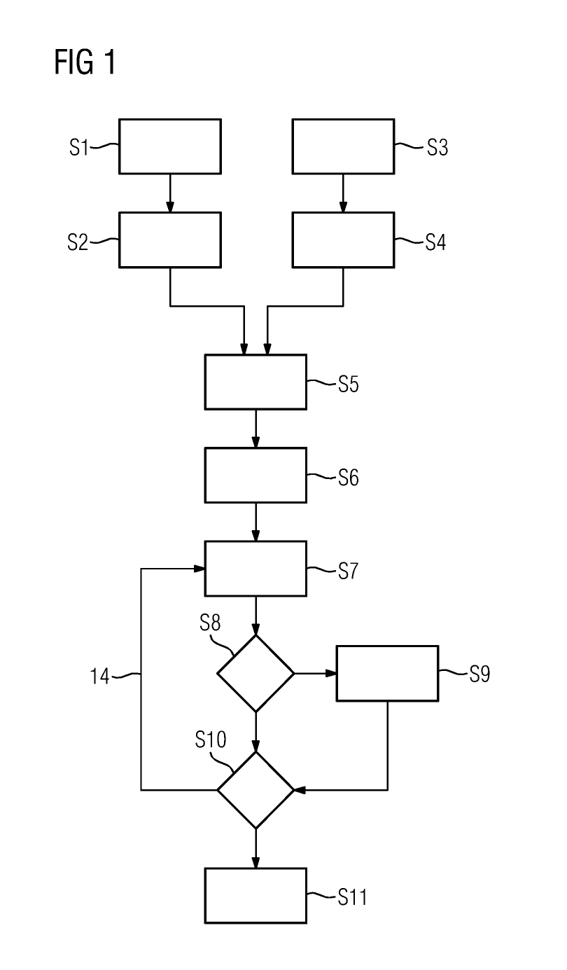

### DETAILED DESCRIPTION

An exemplary embodiment of the method is set forth in greater detail below for the treatment case of an intended minimally invasive intervention in the blood vascular system of the brain of a patient, e.g., the target region here is the brain or the part of a brain of the patient. When preparing for the minimally invasive intervention, the patient is first positioned on a patient table in such a way that sensor elements of a sensor arrangement of a multichannel near-infrared spectroscopy device are as far as possible adjacent to the regions of interest of the brain of the patient. The sensor elements have transmitters and/or receivers for near-infrared radiation, wherein a sensor element may contain a transmitter and a receiver or a sensor element may contain only a transmitter or a receiver in each case. In the present embodiment, the sensor elements are embedded in a head shell of the patient couch, such that their relative geometric arrangement is known. The patient may be held in place in the current position, in order as far as possible to prevent movement. The head of the patient is now situated not only within the head shell, but also in the center of rotation of a C-arm of an X-ray device on which an X-ray detector and an X-ray source are arranged.

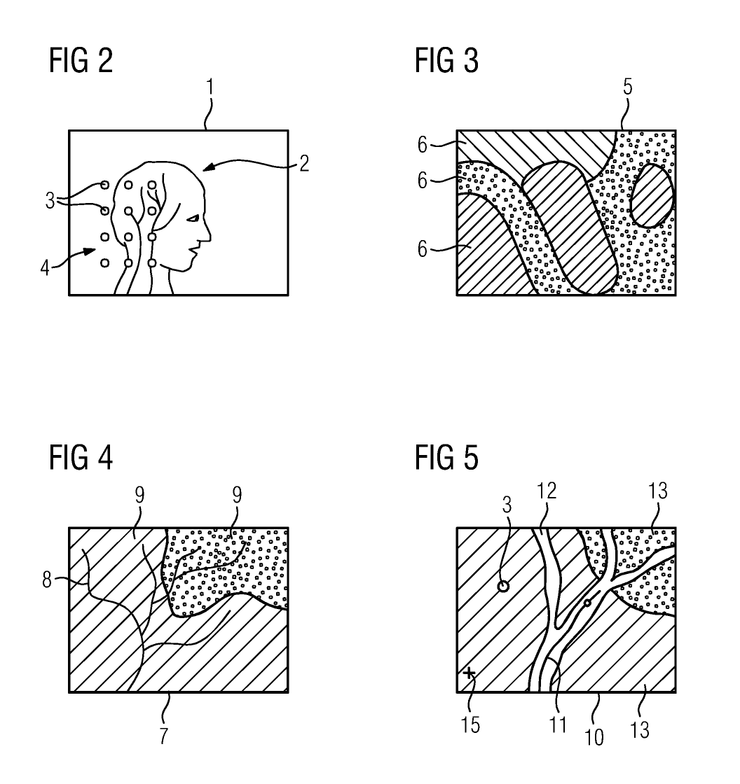

In act S1, a three-dimensional anatomical image data record of the head of the patient is recorded using the X-ray device. In order to achieve this, the C-arm is rotated about the patient, such that two-dimensional projection images of the head may be recorded from different projection directions, and may be used as a basis for constructing a three-dimensional anatomical image data record in a manner that is known, e.g., by filtered rear projection or the like. Since the actual sensor elements significantly attenuate the x-radiation, they may be identified in the projection images and therefore also in the anatomical image data record. FIG. 2 schematically depicts an example of such an anatomical image data record 1. Both the anatomy 2 of the head of the patient and the sensor elements 3 of the sensor arrangement 4 are clearly identifiable.

On the basis of the readily identifiable sensor elements 3, it is possible in act S2, using a segmentation algorithm based on threshold values and also makes use of prior knowledge about the sensor elements 3, to determine in real terms their attenuation characteristics, their geometry, and their spatial arrangement relative to each other, and the position of the sensor elements 3 in the system of coordinates of the X-ray device and hence of the anatomical image data record.

Assuming a patient whose position has not changed in comparison with the recording of the anatomical image data record in act S1, and a sensor arrangement 4 whose position is likewise unchanged, provision is now made in act S3 for recording near-infrared spectroscopy data using the multichannel near-infrared spectroscopy device using the sensor arrangement 4. Since the received near-infrared spectroscopy data may be assigned to the receiving sensor elements 3, it represents the spatial reference points for processing the near-infrared spectroscopy data to produce a near-infrared spectroscopy map, this occurring in act S4. Such a near-infrared spectroscopy map may be one-dimensional or two-dimensional, this depending in particular on the accuracy with which the spatial arrangement of the sensor elements 3 is known in advance. In particular, however, their proximity relationship and approximate arrangement is known in order that the sensor elements that are actually present may also subsequently be assigned to the segmented sensor elements 3 of the anatomical image data record 1.

An example of a near-infrared spectroscopy map 5 is represented schematically in FIG. 2. The near-infrared spectroscopy data, which is spatially assigned therein on the basis of the recording sensor elements 3, describes the cortical oxygenation in the brain of the patient, which may be depicted using color codes, for example, this being symbolized here by the variously shaded regions 6. It may be seen that the near-infrared spectroscopy map 5 does not contain any information relating to the anatomy 2.

In order to allow the near-infrared spectroscopy map to be linked to the anatomical image data record 1, and thus create a registration between the system of coordinates of the X-ray device and the system of coordinates of the near-infrared spectroscopy map 5, the latter system of coordinates being based on assumptions if necessary, use is simply made of the fact that positions of the sensor elements 3 in the anatomical image data record 1 are known as a result of the segmentation in act S2, and at the same time represent the basis on which the near-infrared spectroscopy map 5 is produced, such that the positions of the sensor elements 3 are known in the system of coordinates underlying the near-infrared spectroscopy map 5. If the segmented sensor elements 3 and the sensor elements that record the near-infrared spectroscopy data and provide the basis for the map 5 may now be assigned to each other, it is possible to derive a transformation formula that transfers data from the system of coordinates of the near-infrared spectroscopy map 5 into that of the anatomical image data record 1 and vice versa. A registration is therefore established that does not have to be rigid, particularly if the relative arrangement of the sensor elements 3 is not exactly known by the near-infrared spectroscopy device. This assignment and registration takes place in act S5.

While still preparing for the minimally invasive intervention, the registration may be used in act S6 to determine and represent a combined data record by at least partially amalgamating the near-infrared spectroscopy map 5 with the anatomical image data record 1. Since it will often be a question of assigning the near-infrared spectroscopy data to the anatomy 2, it is appropriate in this case to remove the sensor elements 3 from the anatomical image data record 1 in a known manner before the amalgamation, and thereby prevent them from having a detrimental influence. Since the sensor elements 3 are situated outside of the brain, these portions may usefully and easily be removed from the view for the purposes of the amalgamation. The combined data record may be displayed on a suitable display appliance, e.g., a monitor of the X-ray device.

FIG. 4 schematically depicts an example of such a combined data record 7. A vascular tree 8 may be identified as part of the anatomy 2, and is superimposed by near-infrared spectroscopy data that is again represented by color coding; cf. the regions 9. For example, it may be seen here that less oxygenation and hence less flow of blood is present in the region at the top right-hand side of FIG. 4. The vascular blockage to be removed may be there, for example.

It may be noted at this point that, if the anatomical data record 1 is or may be registered with further image data records, e.g., preoperative magnetic resonance image data records, it is obviously possible for information from these further preoperative image data records to be likewise included in the combined data record 7.

The intervention now begins and image monitoring of this minimally invasive intervention is required, also providing, in some embodiments, functional information relating to the flow of blood. In order to achieve this, act S7 makes provision for recording fluoroscopic images using the X-ray device during the minimally invasive intervention, e.g., two-dimensional X-ray images at low X-ray exposure, while at the same time also regularly recording new near-infrared spectroscopy data and hence near-infrared spectroscopy maps. The fluoroscopic images may be used in this case to display the position of an instrument that is used for the minimally invasive intervention, e.g., a catheter, while the current further near-infrared spectroscopy data may allow the treatment progress to be observed in real time. To this end, it is appropriate to generate an information representation using near-infrared spectroscopy data and data of the fluoroscopic images, anatomical image data of the anatomical image data record 1 being usefully included therein. It is possible to establish such an information representation because the fluoroscopic images are also recorded using the X-ray device, and therefore a registration of the fluoroscopic images with the anatomical image data record 1 is already available and consequently the registration between the anatomical image data record 1 and the system of coordinates of the near-infrared spectroscopy maps 5 may be transferred to the fluoroscopic images.

An exemplary information representation 10 is schematically represented in FIG. 5. This depicts the instrument 11 from the fluoroscopic images, a vascular structure 12 as part of the anatomy 2 from the anatomical image data record 1, and near-infrared spectroscopy data superimposed in color as indicated by the regions 13.

The fluoroscopic image itself was used as a basis for the information representation 10 in FIG. 5. The other structures/color codings are superimposed. Therefore it may also be seen in FIG. 5 that a sensor element 3 from the fluoroscopic image may be identified in the represented section of the information representation 10. Since the sensor elements 3 are expected at a specific position in the fluoroscopic image as a result of the registration, it is possible here to perform a check for possible registration errors caused by movements, in particular movements of the patient, this occurring in act S8. Here the sensor elements 3 are again segmented in the fluoroscopic images and a comparison is made with the reference positions from the registration. If a deviation occurs that exceeds a threshold value, the method continues in act S9, where the registration is corrected in accordance with the movement. This provides that the registration is updated on the basis of the current data for the position of the sensor elements 3. Provision may also be made for a second threshold value (not represented here), which describes a deviation that is too great and initiates the output of a warning notification to a user.

If an update of the registration is not necessary following the check in act S8, the method continues in act S10. Here, a check is performed to determine whether the image monitoring of the minimally invasive intervention may be terminated. If this is not the case, the method continues as per arrow 14 in act S7 with the recording of new data in order to provide that the information representation 10 remains current.

It may nonetheless be noted at this point that cases are readily conceivable in which the sensor elements 3 are not visible in the fluoroscopic images. In this case, provision may be made for at least one marker 15 on a mounting for the sensor arrangement (e.g., the head shell in the present example) in a recorded region, the marker 15 being represented schematically in FIG. 5 and having a fixed spatial relationship to the sensor arrangement. The check in act S8 may then take place on the basis of the expected position of this at least one marker 15.

In act S11, it is still possible to perform a final view after termination of the minimally invasive intervention. For example, further image data records may be recorded as control recordings using the X-ray device, and combined representations generated in conjunction with near-infrared spectroscopy data. Such control recordings may take the form of perfusion measurements and/or recordings of the digital subtraction angiography, for example.

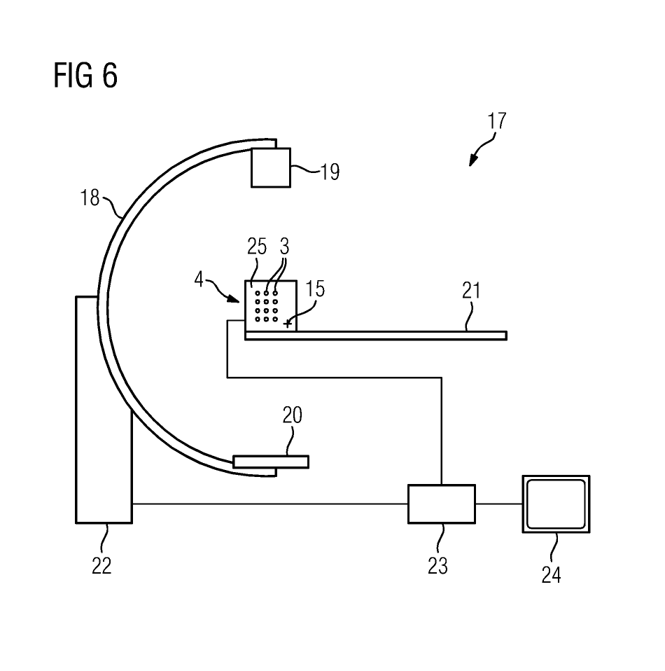

Finally, FIG. 6 depicts a schematic diagram of an X-ray device 17, into which the multichannel near-infrared spectroscopy device is integrated. The X-ray device 17 has a C-arm 18 on which an X-ray source 19 and an X-ray detector 20 are arranged facing each other. The C-arm 18 is designed in such a way that it may be swiveled about a patient couch 21 in order to allow X-ray images to be recorded from different projection directions. It is supported by a suitable stand 22, on which the pivot bearing is also realized. Data recorded by the X-ray detector 20 is transferred to a control device 23, which is only represented schematically here, where the corresponding image data records are created and may then be represented on a display appliance 24, for example.

A head shell 25 is provided on the patient couch 21 as a mounting for the sensor arrangement 4. Data from the sensor elements 3 of the sensor arrangement 4 are likewise transferred to the control device 23, which is therefore also designed for the purpose of establishing near-infrared spectroscopy maps. The integration described above is thereby achieved. The cited markers 15 may also be provided on the head shell 25.

The control device 23 is designed to perform the method, providing that it may activate the components of the X-ray device 17 in order to record X-ray image data and/or near-infrared spectroscopy data, establish image data records and near-infrared spectroscopy maps therefrom, undertake a registration on the basis of the positions of the sensor elements 3, establish information representation and combined data records, etc. Suitable reconstruction units, registration units, representation units, and the like may be provided for this purpose.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for registering a near-infrared spectroscopy map and an anatomical image data record of a target region of a human body, the method comprising:
recording a near-infrared spectroscopy data record of the target region using a multichannel near-infrared spectroscopy device comprising a plurality of sensor elements in a sensor arrangement and using spatial information relating to the sensor elements and an assignment of near-infrared spectroscopy data to the sensor element that recorded the near-infrared spectroscopy data record;
analyzing the near-infrared spectroscopy data record to produce a near-infrared spectroscopy map,
recording a three-dimensional anatomical image data record using an X-ray device without moving either sensor arrangement or target region in relation to the recording of the near-infrared spectroscopy data record, wherein at least some of the sensor elements are visible in the three-dimensional anatomical image data record;
segmenting and localizing the sensor elements in the three-dimensional anatomical image data record; and
registering the near-infrared spectroscopy map and the three-dimensional anatomical image data record on the basis of known positions of the sensor elements relative to the near-infrared spectroscopy map and in the anatomical image data record.

2. The method as claimed in claim 1, wherein a combined data record is established and represented by at least partially amalgamating the near-infrared spectroscopy map with the three-dimensional anatomical image data record.

3. The method as claimed in claim 1, wherein the target region comprises a brain of a patient and the near-infrared spectroscopy map is an oxygenation map.

4. The method as claimed in claim 1, wherein the segmentation of the sensor elements is based on threshold values, makes use of prior knowledge, or is both based on the threshold values and makes use of the prior knowledge, wherein the prior knowledge relates to one or more of the following: geometry characteristics, attenuation characteristics, or a relative arrangement of the sensor elements.

5. The method as claimed in claim 1, wherein image monitoring is performed for a minimally invasive intervention in the patient,
wherein two-dimensional fluoroscopic images of the target region are recorded by the X-ray device, and
wherein, with reference to the registration between the three-dimensional anatomical image data record and the near-infrared spectroscopy map, a registration between the near-infrared spectroscopy map and the fluoroscopic images is established for a combined information representation and at least some of the near-infrared spectroscopy data is superimposed on the fluoroscopic image in the information representation.

6. The method as claimed in claim 5, wherein further recordings of further current near-infrared spectroscopy data are made repeatedly using the sensor arrangement, and the current near-infrared spectroscopy data is used in the information representation in each respective recording.

7. The method as claimed in claim 5, wherein at least some of the anatomical image data is also used in the information representation.

8. The method as claimed in claim 5, wherein the sensor elements are also segmented in the fluoroscopic image and the sensor elements are analyzed with regard to registration errors caused by movement.

9. The method as claimed in claim 8, wherein either or both an automatic correction of the registrations is performed on the basis of the sensor elements that are segmented in the fluoroscopic image or a warning notification is output to a user if a deviation from the registration exceeds a threshold value.

10. The method as claimed in claim 1, wherein the three-dimensional anatomical image data record is registered with a previously recorded image data record or an image data record that has been recorded, and
wherein a registration between the further image data record and the near-infrared spectroscopy map is established using the registration between the three-dimensional anatomical image data record and the near-infrared spectroscopy map.

11. The method as claimed in claim 11, wherein the further image data record is recorded using the X-ray device and comprises an image data record of the digital subtraction angiography, a perfusion image data record, or digital subtraction angiography and perfusion image data record.

12. The method as claimed in claim 1, wherein a four-dimensional near-infrared spectroscopy map is established as a result of recording near-infrared spectroscopy data at various time points.

13. The method as claimed in claim 1, wherein the X-ray device comprises an integrated near-infrared spectroscopy sensor arrangement.

14. The method as claimed in claim 13, wherein the sensor arrangement is arranged in a known spatial relationship to an X-ray source and an X-ray detector of the X-ray device by a designated fixing the X-ray device.

15. The method as claimed in claim 13, wherein the registration of the systems of coordinates of the X-ray device and the near-infrared spectroscopy maps determined by the sensor arrangement is established in context of a calibration measurement and used for subsequent diagnostic measurements.

16. The method as claimed in claim 13, wherein at least one marker, which is visible in the three-dimensional anatomical image data record, is arranged in a fixed geometrical relationship to the sensor elements and is taken into consideration when establishing the registration between the three-dimensional anatomical image data record and the near-infrared spectroscopy map.

17. The method as claimed in claim 16, wherein the fixed geometrical relationship comprises arranging the at least one marker on a mounting for the sensor arrangement.

18. An X-ray device comprising:
a control device, wherein the control device is configured to:
(1) record a near-infrared spectroscopy data record of the target region using a multichannel near-infrared spectroscopy device comprising a plurality of sensor elements in a sensor arrangement and using spatial information relating to the sensor elements and an assignment of near-infrared spectroscopy data to the sensor element that recorded the near-infrared spectroscopy data record;
(2) analyze the near-infrared spectroscopy data record to produce a near-infrared spectroscopy map,
(3) record a three-dimensional anatomical image data record using the X-ray device without moving either sensor arrangement or target region in relation to the recording of the near-infrared spectroscopy data record, wherein at least some of the sensor elements are visible in the three-dimensional anatomical image data record;
(4) segmenting and localizing the sensor elements in the three-dimensional anatomical image data record; and
(5) registering the near-infrared spectroscopy map and the three-dimensional anatomical image data record on the basis of known positions of the sensor elements relative to the near-infrared spectroscopy map and in the anatomical image data record.

19. The X-ray device as claimed in claim 18, wherein a near-infrared spectroscopy device with a sensor arrangement is integrated therein.

20. The X-ray device as claimed in claim 18, wherein the X-ray device comprises a C-arm on which an X-ray source and an X-ray detector are arranged facing each other.
